(19) United States
(12) Patent Application Publication
(10) Pub. No.: US 2024/0310381 A1
(43) Pub. Date: Sep. 19, 2024

(54) TRANSGENIC ANIMAL IN WHICH APEX2 IS SPECIFICALLY EXPRESSED IN MITOCHONDRIAL MATRIX AND USES THEREOF

(71) Applicants: SEOUL NATIONAL R&BD FOUNDATION, Seoul (KR); KOREA ADVANCED INSTITUTE OF SCIENCE AND TECHNOLOGY, Daejeon (KR)

(72) Inventors: Hyun Woo RHEE, Seoul (KR); Jae Myoung SUH, Daejeon (KR); Jong-Seo KIM, Seoul (KR); Isaac PARK, Seoul (KR); Kwang-eun KIM, Daejeon (KR)

(73) Assignees: SEOUL NATIONAL R&BD FOUNDATION, Seoul (KR); KOREA ADVANCED INSTITUTE OF SCIENCE AND TECHNOLOGY, Daejeon (KR)

(21) Appl. No.: 18/575,123

(22) PCT Filed: Jun. 24, 2022
(86) PCT No.: PCT/KR2022/009012

§ 371 Date: Dec. 28, 2023

(30) Foreign Application Priority Data
Jun. 28, 2021 (KR) 10-2021-0084274

(51) Int. Cl. G01N 33/68; A01K 67/0275; C12N 9/08; C12N 15/85

(52) U.S. Cl. CPC G01N 33/6848; A01K 67/0275; C12N 9/0065; C12N 15/8509; A01K 2227/105; C07K 2319/07; C12Y 111/01011

(57) ABSTRACT

The present invention relates to a transgenic animal in which APEX2 is specifically expressed in a mitochondrial matrix and to uses thereof. When the transgenic animal according to the present invention is used, it is possible to overcome the limitation of being susceptible to contamination with proteins derived from other organelles or other tissue cells in existing mitochondrial proteomic studies. Accordingly, mitochondrial matrix proteomic research with a high reliability is possible, tissue-specific mitochondrial matrix proteomics can easily be analyzed, and a wide range of applications are possible in basic life science research, drug development, and diagnostic research related to mitochondrial proteomics.

### TECHNICAL FIELD

The present invention relates to a transgenic animal in which APEX2 is specifically expressed in the mitochondrial matrix and to uses thereof, and more specifically, to a transgenic animal in which APEX2 is specifically expressed in the mitochondrial matrix and may be utilized for tissue-specific mitochondrial proteomic research and drug target protein identification in mitochondria using a proximity labeling method.

### BACKGROUND ART

Mitochondria are central cellular organelles which not only produce ATP, which is the energy source of cells, but also produce molecules such as lipid molecules, heme, and pyrimidine, and are responsible for apoptosis and antiviral signaling. Studies conducted by many researchers have found that when mitochondrial dysfunction occurs, abnormalities in mitochondrial proteins become an important pathogenic factor not only for genetic diseases such as Leber's hereditary optic neuropathy, but also for neurological diseases such as Alzheimer's disease and Parkinson's disease. However, it is still quite unclear what kind of molecular association the mitochondrial proteins predicted to be associated with each disease specifically have. This is because it is still not understood how proteins known to be present in mitochondria are distributed while having what type of organic relationship with large proteomic components and the mechanism for important mitochondrial biogenesis has not been completely elucidated.

Since mitochondria are cellular organelles that produce ATP, which is the bioenergy molecule, protein research began by biochemists in the early and mid-20th century, when biochemical research began. In particular, protein components that mediate the TCA cycle in the mitochondrial matrix, OxPhos complexes that mediate the oxidative phosphorylation process in mitochondria, and the like have been intensively studied for more than half a century, and in the 21st century, a total of 1,200 proteins (Mitocarta) present in mitochondria were identified through mass spectrometry by isolating mitochondria. Although research has been intensively conducted on mitochondrial proteomes, it cannot be said that mitochondrial proteomes have been completely understood.

Existing research on mitochondrial proteomes has mainly been conducted by isolating mitochondria using a centrifuge and then analyzing the proteins. However, when mitochondrial proteins are analyzed using this method, there is a limitation in that they are often contaminated with cytoplasmic proteins ejected from other organelles or dead cells, making it impossible to accurately analyze only mitochondrial proteins. Further, since mitochondria have a double-membrane structure, existing experiments cannot reveal information on the detailed location of proteins in mitochondria.

Meanwhile, although recent proteomic studies have advanced to the level where it is possible to unbiasedly identify proteins present in a sample injected into a mass spectrometer, the problem is that many false-positive proteins generated when a sample to be injected into a mass spectrometer has not been properly prepared in the previous experimental process are also analyzed, resulting in an analysis result that is not very useful to the experimenter. In order to perform mass spectrometry experiments on proteins present in macromolecular complexes, an ‘isolation’ process in which organelles or macromolecular complexes desired by researchers are purified from living cells is required, and in order to typically isolate a specific organelle, a process of isolating a specific organelle is generally performed by obtaining a fraction corresponding to the density of the specific organelle while performing a concentration gradient high-speed centrifugation process. In this case, the problem is that fractions isolated based on only density are almost always contaminated with other proteins, and thus when these fractions are injected into a mass spectrometer, not only a proteome corresponding to a large proteome which an experimenter desires, but also proteomes of other macromolecular complexes that are inevitably contaminated during the process are simultaneously analyzed, and eventually, when a protein list obtained as a result of mass spectrometry is examined, the protein list becomes experimental data in which the information on the proteomes of other cellular organelles is mixed, and as a result, it becomes impossible to know accurate information on what type of protein remains in a specific giant proteome. Proximity labeling technology has recently been developed to alleviate these problems.

Two enzymes are most widely used in proximity labeling technology: BioID (biotin protein ligase) and APEX (peroxidase). A peroxidase is an enzyme protein having a heme group, and it is known that when peroxidases react with hydrogen peroxide, the peroxidases have a Compound I/II state with strong oxidizing power and take electrons from phenolic compounds to generate phenoxyl radicals. Currently, the most commonly used peroxidase is horseradish peroxidase (HRP), which is an enzyme derived from horseradish and has very high reactivity, so that in the biological field, horseradish peroxidase has been utilized in a technique called tyramide signal amplification assay (TSA) using a fluorescent signal obtained by labeling tyrosine groups of surrounding proteins with a plurality of probes using phenoxy radicals which are generated from peroxidase. Since the lifetime of radical materials in an aqueous solution is shorter than several milliseconds, radicals generated from peroxidases have a specificity in which it is possible to label only proteins within a radius of 20 nm. Recently, the research team of Professor Alice Ting of MIT (USA) has developed a new method capable of mapping a local proteome by generating a phenoxy radical to which biotin is attached in a cellular organelle in situ using APEX (engineered ascorbate peroxidase), which does not lose its activity no matter where it is expressed in cells, and reported a result of mapping the proteome of the mitochondrial matrix (Rhee H W. et al., Science, 2013, 339, 1328-1331) and the mitochondrial double membrane space (Hung V. et al., Molecular Cell 2014, 55, 332-341). Meanwhile, PEX2 is an enzyme that improves the efficiency of the existing APEX enzyme (Lam, Stephanie S et al., Nature methods, 2015, 12(1):51-4). As a result of intensive studies on a method capable of expanding the scope of use of proximity labeling technology, which has traditionally been used to overcome the limitations of mitochondrial proteomic research methods in the related art, the present inventors confirmed that a transgenic mouse in which APEX2 is specifically expressed in the mitochondrial matrix could be developed, and when such a transgenic mouse was used, a specific mitochondrial matrix protein could be easily labeled and identified, and could be utilized in the identification of a drug-target protein within mitochondria, thereby completing the present invention.

### DISCLOSURE

### Technical Problem

The present invention is directed to providing a transgenic animal that can be used for the tissue-specific analysis of a mitochondrial matrix protein, and a method for identifying a tissue-specific mitochondrial matrix protein using the transgenic animal.

### Technical Solution

One aspect of the present invention provides a fusion protein in which a mitochondrial matrix targeting peptide and a proximity labeling enzyme are fused.

Another aspect of the present invention provides a recombinant expression vector or recombinant RNA including a sequence encoding the fusion protein.

In the present invention, the recombinant expression vector or recombinant RNA may include:
a first nucleotide sequence encoding a mitochondrial matrix targeting sequence:
a second nucleotide sequence encoding a proximity labeling enzyme; and
a promoter to which the first and second nucleotide sequences are operably linked.

In the present invention, the proximity labeling enzyme may be APEX, APEX2 or TurboID.

The present invention also provides a transgenic cell line or fertilized egg for producing a transgenic animal into which the recombinant expression vector or recombinant RNA has been introduced.

Still another aspect of the present invention provides a transgenic animal in which the proximity labeling enzyme is specifically expressed in the mitochondrial matrix.

In the present invention, the animal may be produced by transplanting the transgenic cell line or fertilized egg into the oviduct of a surrogate mother, which is an animal other than a human.

In the present invention, the animal may be a mouse or rat.

Yet another aspect of the present invention provides a method for identifying a tissue-specific mitochondrial matrix protein, the method including the following steps:
(a) expressing a proximity labeling enzyme in the mitochondrial matrix of the tissue in the transgenic animal;
(b) labeling the mitochondrial matrix protein with a phenol probe through a chemical reaction caused by a proximity labeling enzyme expressed in the mitochondrial matrix in the tissue of the transgenic animal; and
(c) identifying the labeled protein as a tissue-specific mitochondrial matrix protein.

In the present invention, the proximity labeling enzyme may be APEX, APEX2 or TurboID.

In the present invention, in step (b), the tissue may be isolated, and then subsequently treated with any one reagent selected from the group consisting of biotin phenol, desthiobiotin phenol, alkyne-phenol and azide-phenol, and hydrogen peroxide to label the mitochondrial matrix protein with a phenol probe.

In the present invention, step (c) may be characterized in that the labeled protein is isolated and identified using streptavidin beads.

In the present invention, the protein labeled in step (c) may be identified by mass spectrometry.

Still yet another aspect of the present invention provides a method for identifying a target protein in a drug-specific mitochondrial matrix, the method including the following steps:
(a) administering a drug to the transgenic animal;
(b) expressing a proximity labeling enzyme in the mitochondrial matrix of the tissue in the drug-administered animal;
(c) labeling a mitochondrial matrix protein with a phenol probe through a chemical reaction caused by a proximity labeling enzyme expressed in the mitochondrial matrix in the tissue of the transgenic animal; and
(d) when the labeled protein has a significant change in expression level compared to a control, identifying the protein as a drug-specific mitochondrial matrix target protein.

In the present invention, the proximity labeling enzyme may be APEX, APEX2 or TurboID.

In the present invention, in step (b), the tissue may be isolated, and then subsequently treated with any one reagent selected from the group consisting of biotin phenol, desthiobiotin phenol, alkyne-phenol and azide-phenol, and hydrogen peroxide to label the mitochondrial matrix protein with a phenol probe.

In the present invention, step (d) may be characterized in that the labeled protein is isolated and identified using streptavidin beads.

In the present invention, the protein labeled in step (c) may be identified by mass spectrometry.

### Advantageous Effects

When the transgenic animal according to the present invention is used, it is possible to overcome the limitation of being susceptible to contamination with proteins derived from other organelles or other tissue cells in existing mitochondrial proteomic studies. Accordingly, mitochondrial matrix proteomic research with high reliability is possible, tissue-specific mitochondrial matrix proteomes and target proteins in a drug-specific mitochondrial matrix can easily be analyzed, and a wide range of applications are possible in basic life science research, drug development, and diagnostic research related to mitochondrial proteomics.

### DESCRIPTION OF DRAWINGS

FIG. 1 is a schematic view showing an exemplary embodiment of the present invention in which a tissue-specific mitochondrial matrix protein is labeled with biotin using a transgenic mouse transfected with MTS-V5-APEX2, and then isolated using streptavidin beads, and then a mitochondrial matrix proteome is selectively analyzed by LC-MS/MS analysis.

FIG. 2 is a schematic view showing the process of identifying a tissue-specific mitochondrial proteome according to an exemplary embodiment of the present invention.

FIG. 3 shows the results of comparing information on mitochondrial matrix proteins in muscle and the heart identified according to an exemplary embodiment of the present invention.

FIG. 4 shows the results of tissue-specific mitochondrial TEM images taken according to an exemplary embodiment of the present invention.

FIG. 5 shows information on mitochondrial matrix proteins in myofiber cells of muscle obtained by crossing floxed MAX-Tg mice with Myf5-Cre.

### MODES OF THE INVENTION

Unless otherwise defined, all technical and scientific terms used herein have the same meaning as commonly understood by a person with ordinary skill in the art to which the present invention pertains. In general, the nomenclature used herein and the experimental methods described below are those well-known and typically used in the art.

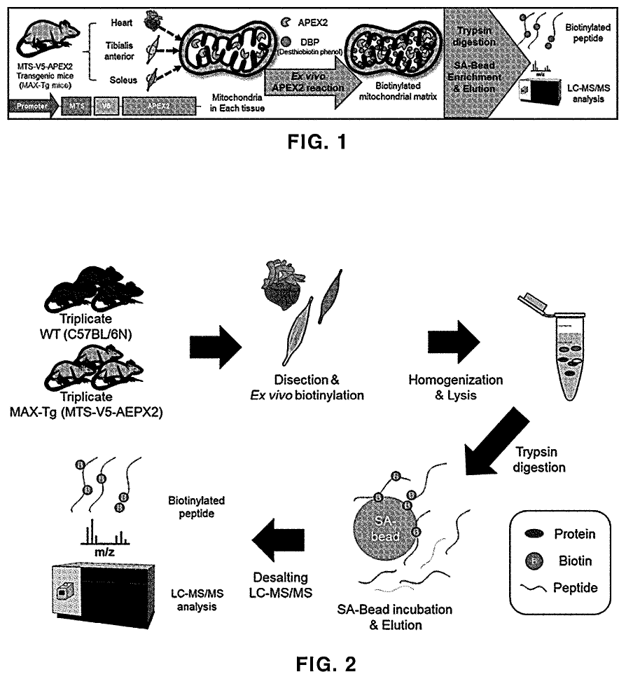

Proximity labeling technology is a technology that can selectively label only a protein distributed in a specific space within living cells with biotin, isolate the biotin-labeled proteins with streptavidin beads, and then identify the isolated proteins using a mass spectrometer, and in the present invention, a transgenic mouse (hereinafter used interchangeably with ‘MAX-Tg mouse’) capable of labeling and identifying a mitochondrial protein in a specific tissue was developed by expressing the APEX2 enzyme, which is one of the proximity labeling techniques, together with a mitochondrial targeting sequence such that it is specifically expressed in the mouse mitochondrial matrix (see FIGS. 1 and 2).

Therefore, in one aspect, the present invention relates to a fusion protein in which a mitochondrial matrix targeting peptide and a proximity labeling enzyme are fused.

In the present invention, the fusion protein may include an amino acid sequence of SEQ ID NO: 1 and an amino acid sequence of SEQ ID NO: 3, but is not limited thereto.

In the present invention, the mitochondrial matrix targeting peptide may have the amino acid sequence represented by SEQ ID NO: 1, but is not limited thereto.

In the present invention, the proximity labeling enzyme may have the amino acid sequence represented by SEQ ID NO: 3, but is not limited thereto.

Further, in another aspect, the present invention relates to a recombinant expression vector or recombinant RNA including a sequence encoding the fusion protein.

In the present invention, the recombinant expression vector or recombinant RNA may include:
a first nucleotide sequence encoding a mitochondrial matrix targeting sequence;
a second nucleotide sequence encoding a proximity labeling enzyme; and
a promoter to which the first and second nucleotide sequences are operably linked. In the present invention, the proximity labeling enzyme may be APEX, APEX2 or TurboID, but is not limited thereto.

In still another aspect, the present invention relates to a transgenic cell line or fertilized egg for producing a transgenic animal into which the recombinant expression vector or recombinant RNA has been introduced.

In yet another aspect, the present invention relates to a transgenic animal in which the proximity labeling enzyme is specifically expressed in the mitochondrial matrix.

In the present invention, the animal may be produced by transplanting the transgenic cell line or fertilized egg into the oviduct of a surrogate mother, which is an animal other than a human.

In the present invention, the animal may be a mouse or a rat, but is not limited thereto, and various animals such as a goat, a rabbit, and a monkey may be used. The animal may be, for example, a mammal.

In the present invention, the recombinant expression vector or recombinant RNA includes DNA or RNA known in the art, which has been arbitrarily manipulated such that the proximity labeling enzyme can be specifically expressed in the mitochondrial matrix in the transgenic animal.

The transgenic mouse according to the present invention enables the mechanism of action of each tissue and organ to be observed at the molecular level by selectively isolating tissues to be analyzed and observing the expression pattern of mitochondrial matrix proteins, and when crossed with other disease model mice, may be usefully utilized for research on disease pathogenesis according to changes in mitochondrial matrix proteins because it is possible to observe the mitochondrial matrix proteins in the disease model.

For example, muscles are very important organs for energy metabolism in the body, and muscle cells are one of the cells with the largest distribution of mitochondria, which are the core organelles of life phenomena. Therefore, when a mitochondrial matrix proteome in muscle tissue is analyzed using the transgenic mouse according to the present invention, there is an advantage in that it is possible to understand the disease mechanism at the molecular level regarding which muscle-specific mitochondrial matrix proteins are deleted in muscle mitochondria-related diseases such as muscle loss, myopathy, myasthenia, and muscular atrophy to cause the corresponding disease, which was not understood in the related art.

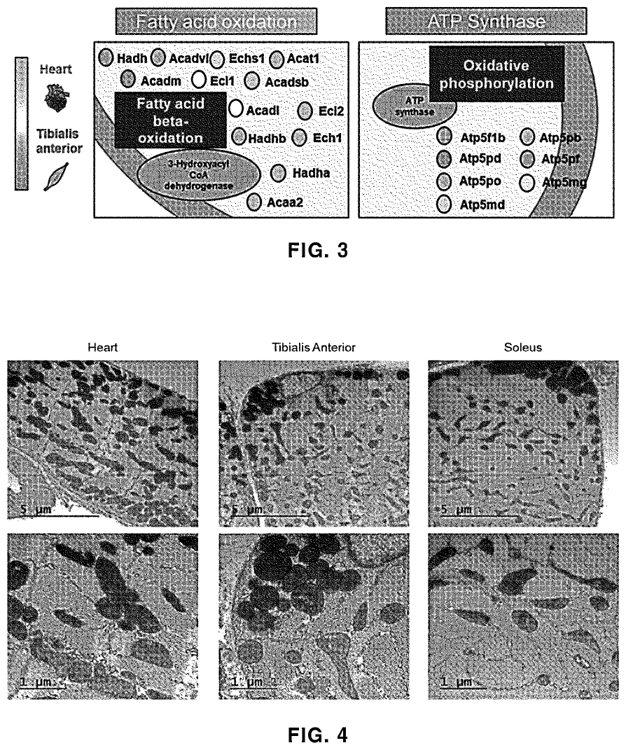

In one embodiment, in the present invention, the heart and muscle tissues of the transgenic mouse were isolated to label a mitochondrial matrix protein with biotin in an ex vivo environment, the labeled protein was isolated using streptavidin beads, and it was possible to identify mitochondrial matrix proteins specifically expressed in the heart and muscle, respectively, by subjecting the labeled protein to mass spectrometry (see FIG. 3).

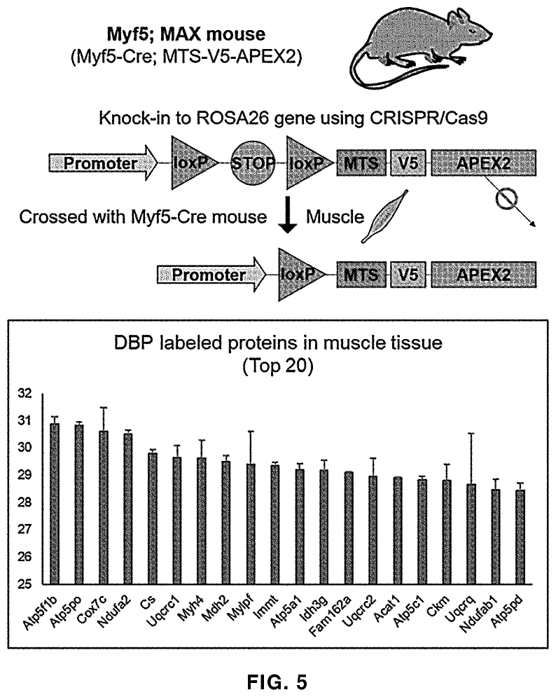

Further, among the 200 proteins in the tibialis anterior muscle, a list of the top 20 most abundant proteins was confirmed, and it could be confirmed that the majority of the proteins are proteins in an OXPHOS complex (Atp5h, Atp5o, Atp5b, Atp5c1, Atp5a1, Ndufab1, Ndufa2, Cox7c, Uqcrc1, and Uqcrq) or the TCA cycle process (Idh3g, Mdh2, and Cs) (see FIG. 5).

Therefore, in yet another aspect, the present invention relates to a method for identifying a tissue-specific mitochondrial matrix protein, the method including the following steps:
(a) expressing a proximity labeling enzyme in the mitochondrial matrix of the tissue in the transgenic animal;
(b) labeling a mitochondrial matrix protein with a phenol probe through a chemical reaction caused by a proximity labeling enzyme expressed in the mitochondrial matrix in the tissue of the transgenic animal; and
(c) identifying the labeled protein as a tissue-specific mitochondrial matrix protein.

In the present invention, the proximity labeling enzyme may be APEX, APEX2 or TurboID, but is not limited thereto.

In the present invention, step (b) may be characterized in that the tissue is isolated, and then subsequently treated with any one reagent selected from the group consisting of biotin phenol, desthiobiotin phenol, alkyne-phenol and azide-phenol, and hydrogen peroxide to label the mitochondrial matrix protein with a phenol probe, but is not limited thereto.

In the present invention, the method may further include digesting the protein with trypsin after step (b).

In the present invention, step (c) may be characterized in that the labeled protein is isolated and identified using streptavidin beads.

In the present invention, the protein labeled in step (c) may be identified by mass spectrometry.

In the present invention, the tissue may be muscle tissue or heart tissue, but is not limited thereto.

In the present invention, changes in the mitochondrial matrix may also observed specifically in tissue or before and after induction of a specific disease using an electron microscope (see FIG. 4).

In the present invention, a target protein in the mitochondrial matrix of a drug may also be identified by observing changes in the expression level of the protein in the mitochondrial matrix caused by treatment with the drug.

Therefore, in yet another aspect, the present invention relates to a method for identifying a target protein in a drug-specific mitochondrial matrix, the method including the following steps:
(a) administering a drug to the transgenic animal;
(b) expressing a proximity labeling enzyme in the mitochondrial matrix of the tissue in the drug-administered animal;
(c) labeling a mitochondrial matrix protein with a phenol probe through a chemical reaction caused by a proximity labeling enzyme expressed in the mitochondrial matrix in the tissue of the transgenic animal; and
(d) when the labeled protein has a significant change in expression level compared to a control, identifying the protein as a drug-specific mitochondrial matrix target protein.

In the present invention, the proximity labeling enzyme may be APEX, APEX2 or TurboID, but is not limited thereto.

In the present invention, step (c) may be characterized in that the tissue is isolated, and then subsequently treated with any one reagent selected from the group consisting of biotin phenol, desthiobiotin phenol, alkyne-phenol and azide-phenol, and hydrogen peroxide to label the mitochondrial matrix protein with a phenol probe, but is not limited thereto.

In the present invention, step (d) may be characterized in that the labeled protein is isolated and identified using streptavidin beads, but is not limited thereto.

In the present invention, the protein labeled in step (d) may be identified by mass spectrometry, but is not limited thereto.

In the present invention, the control may refer to a group in which transgenic animals are subjected to the same process as animals treated with a drug without being treated with the drug.

In the present invention, the significant change in expression level may be evaluated as a significant change in expression level when there is an increase or decrease in expression level by about 10% or more, for example, 20%, 30%, 40%, 50%, 60%, 70%, 80%, 90% or about 100%, compared to the control.

As mentioned herein, the term “promoter” refers to a DNA sequence that, when linked to a specific sequence, can regulate the transcription of a specific nucleotide sequence into mRNA, and preferably, is a proximal promoter or distal promoter.

As mentioned herein, the term “recombinant vector” refers to a genetic construct that includes an essential regulatory element operably linked to allow expression of a genetic insert as a vector capable of expressing a target protein or target RNA in a suitable host cell.

In the present invention, a mitochondrial matrix targeting sequence and a proximity labeling enzyme may be operably linked to a promoter.

The recombinant vector is preferably linear DNA, plasmid DNA, or a recombinant viral vector, but is not limited thereto. In addition, the recombinant viral vector may be a retrovirus, an adenovirus, a Herpes simplex virus, and a lentivirus, but is not limited thereto.

A proximal promoter refers to a portion within about 250 base pairs forward from the transcription start site, is the main portion that directly affects the regulation of transcription, and is a site to which specific transcription regulatory factors bind.

A distal promoter is located further forward from the transcription start site, generally has a weaker influence than the proximal promoter and plays an auxiliary role in regulating transcription, and becomes a site to which specific transcriptional regulatory factors bind.

In the present invention, a transgenic animal may be defined as an animal that has acquired new genetic traits not through traditional breeding but through a recombinant DNA technology and a germ cell engineering method. That is, the transgenic animal means that the gene a of an animal called A is not in an animal called B, but the traits of gene a, that is, the ability, is allowed to be expressed in B by directly passing the gene a on to the B animal without going through the process of mating using the recombinant DNA technology and the germ cell engineering method. There are two main types of such transgenesis: somatic cell transgenesis and germ cell transgenesis. Somatic cell transgenesis refers to a case in which a newly acquired genetic trait appears in the animal but is not passed on to the next generation. A representative example of such a case is gene therapy in humans. When a disease is caused by an abnormality or deficiency of a specific gene, a normal gene is injected into the cells of a patient to allow them to function normally and be cured, and in this case, the newly introduced gene only functions in the patient's own generation and is not passed on to future generations. In contrast, germ cell transgenesis refers to a case in which a new genetic trait is passed not only on to the present generation but also to future generations, either by directly transferring a new gene to germ cells or by transferring transgenic cells to germ cells. Generally, the production of transgenic animals in a true sense is performed through germ cell transgenesis.

As mentioned herein, the term “transgenesis” refers to changing the genetic properties of an organism by externally supplied DNA. A transgenesis method may be appropriately selected and applied among various methods known in the related art, for example, techniques using microinjection, electroporation, particle bombardment, sperm-mediated gene transfer, viral infection, direct muscle injection, an insulator, and a transposon.

It is preferred that the injection of a vector is performed using microinjection, but the method is not limited thereto. More specifically, the following method may be used in order to inject the vector into fertilized eggs of an animal. First, pronuclear injection is a method in which DNA is microinjected into pronuclei at the one-cell stage or a nucleus is injected into fertilized eggs at the two-cell stage. This method is the safest and most reliable method for gene transfer, and has an advantage in that intraspecific efficiency is consistent despite variation among species, and a gene can be injected regardless of the size of the DNA fragment. However, this method has disadvantages in that the efficiency of acquiring a transformant is low, the quality of the injected DNA should be good due to origination from the gene used during transduction, and the effect varies depending on the location of the chromosome where the gene is inserted. Furthermore, it is important to maintain an appropriate concentration of the DNA to be injected, and as a pronucleus for injecting DNA, it is better to select a male pronucleus, which has a larger size than a female nucleus. When a foreign DNA material is injected into a pronucleus, it is advantageous to inject multiple copies of a gene, called concatamers, rather than to inject a single copy of the gene, because the chance that DNA will fuse increases. The injection of DNA is performed during the DNA synthesis phase (S-phase), which is the period when chromosomes unravel, and in order to increase DNA transfer efficiency, it is possible to use a method of increasing the concentration of DNA to be injected, a method of increasing DNA repair activity, a method of unravelling chromosomes by applying changes in temperature to enable gene insertion, and a method of performing reverse transcription using a retroviral integrase. Next, there is a method of injecting a gene using a viral system. An adenoviral vector, a retroviral vector, or an adeno-associated virus vector may be used, and among them, the retroviral vector is the most commonly used. Retroviruses are single-stranded RNA genes and remain in the form of proviruses in the chromosomes of host cells. Foreign DNA is inserted into this proviral DNA using the reverse transcription function of endogenous retroviruses (ERVs) to form transgenic cells. Using this principle, after collecting fertilized eggs at the four- to eight-cell stage, the zona pellucida is removed, the fertilized eggs are cultured with virus-producing cells for 16 to 24 hours, and then may be transplanted into a surrogate mother to create an animal carrying a foreign gene. This method has characteristics that the efficiency is high: once a gene is inserted into a chromosome, the gene cannot be reversed: the gene can be artificially inserted into the chromosome at a desired location: partial propagation is possible ex vivo; and a catalytic reaction can occur due to a viral enzyme. In addition, this method has an advantage in that it is technically easier than injecting DNA into the pronucleus or nucleus, equipment and operations are simple, and accurate physiological investigation is enabled by inserting a single copy of a target gene. However, this method has disadvantages in that care should be taken when handling the virus in consideration of safety because a retrovirus that is lethal to humans is used, most of the transgenic animals are passed on to the next generation in a mosaic manner due to species specificity and the impossibility of being inserted into initial embryos, and there is a limitation in the size of the introduced gene. Since the virus is encapsulated, the infection efficiency of retroviral vectors is determined depending on the possibility of interaction between the virus and the cell membrane and the success or failure of insertion at the mitotic stage. In addition to this method, cytoplasmic injection of a DNA solution or cytoplasmic injection of a polylysine/DNA mixture may be used, but the method is not limited thereto.

Preferably, in the present invention, a vector may be transformed into mouse or rat fertilized eggs through microinjection.

In the present invention, the term ‘protein’ includes ‘peptide.’

### EXAMPLES

Hereinafter, the present invention will be described in more detail through examples. These examples are only for exemplifying the present invention, and it will be apparent to those of ordinary skill in the art that the scope of the present invention is not interpreted as being limited by these examples. Experiments were performed using products manufactured by Sigma-Aldrich as reagents used in the present invention, unless otherwise stated.

### Example 1. Production of Transgenic (MAX-Tg) Mouse in Which APEX2 is Specifically Expressed in Mitochondrial Matrix

A mitochondrial targeting sequence (MTS) was used such that an APEX2 enzyme could be expressed in the mitochondrial matrix. A transgenic mouse was produced such that the APEX2 enzyme was specifically expressed in the mitochondrial matrix.

The amino acid sequences used are as follows.

<SEQ ID NO: 1: MTS amino acid sequence>
MLATRVFSLVGKRAISTSVCVRAH
<SEQ ID NO: 2: V5 amino acid sequence>
GKPIPNPLLGLDST
<SEQ ID NO: 3: APEX2 amino acid sequence>
GKSYPTVSADYQDAVEKAKKKLRGFIAEKRCAPLMLRLAFHSAGTFDKG
TKTGGPFGTIKHPAELAHSANNGLDIAVRLLEPLKAEFPILSYADFYQL
AGVVAVEVTGGPKVPFHPGREDKPEPPPEGRLPDPTKGSDHLRDVFGKA
MGLTDQDIVALSGGHTIGAAHKERSGFEGPWTSNPLIFDNSYFTELLSG
EKEGLLQLPSDKALLSDPVFRPLVDKYAADEDAFFADYAEAHQKLSELG
FADA
<SEQ ID NO: 4: MTS-V5-APEX2 amino acid sequence>
MLATRVFSLVGKRAISTSVCVRAHKDPGKPIPNPLLGLDSTGKSYPTVS
ADYQDAVEKAKKKLRGFIAEKRCAPLMLRLAFHSAGTFDKGTKTGGPFG
TIKHPAELAHSANNGLDIAVRLLEPLKAEFPILSYADFYQLAGVVAVEV
TGGPKVPFHPGREDKPEPPPEGRLPDPTKGSDHLRDVFGKAMGLTDQDI
VALSGGHTIGAAHKERSGFEGPWTSNPLIFDNSYFTELLSGEKEGLLQL
PSDKALLSDPVFRPLVDKYAADEDAFFADYAEAHQKLSELGFADA*

DNA for microinjection of an MTS-VS-APEX2 vector was prepared as follows. MTS-VS-APEX2 DNA is a sequence fragment (about 2.1 kb) including the restriction enzymes MluI and NaeI and the CMV promoter located before and after a nucleotide sequence encoding the amino acid sequence of SEQ ID NO: 4. MTS-VS-APEX2 Tg mice were generated, crossed, and maintained in pathogen-free conditions at Macrogen, Inc. 5 to 8-week-old female C57BL/6N mice were intraperitoneally injected with gonadotropin (7.5 IU) and human chorionic gonadotropin (hCG; 5 IU) to induce ovulation. After hCG injection, the female mice were mated with C57BL/6N male mice. After fertilized embryos were harvested, MTS-V5-APEX2 DNA was co-microinjected into single-cell embryos. DNA (4 ng/μL) for microinjection was directly injected into the male pronucleus of zygotes using a micromanipulator, and the microinjected embryos were incubated at 37° C. for 1 to 2 hours. Fourteen to sixteen injected single-cell-stage embryos were surgically transplanted into the oviducts of ICR mice. After the F0 offspring were born, genotyping was performed using cut tail samples to detect the presence of the transgene. A PCR experiment was performed using a specific primer pair (F: 5′-GTCGACGAGCTCGTTTAGTGA, R: AAGACCGTTGTTAGCGCTGTG-3′).

### Example 2. Analysis of Mitochondrial Matrix Proteomes in Muscle and Heart Tissues Using MAX to Mice

After muscle and heart tissues were each isolated from the transgenic mouse produced in Example 1, the mice were euthanized after respiratory anesthesia using alfaxalone (Jurox Inc), cardiac blood sampling was performed, and skeletal muscle and heart tissue were dissected and separated using forceps and surgical scissors. The skeletal muscle and heart tissue were washed with saline and cut into pieces about 5 mm³ in size. The cut tissue samples were treated with biotin phenol (Alfa Aesar) at 500 μM for 1 hour in a low-temperature environment on ice, and then treated with hydrogen peroxide (Sigma-Aldrich) at 2 mM for 2 minutes to induce the radical reaction of biotin such that mitochondrial matrix proteins in each tissue were labeled with biotin. Thereafter, the reaction was terminated by adding quenching Buffer (1 M sodium azide, Trolox, and sodium ascorbate) in order to remove radicals. The labeled tissue was homogenized, and then dissolved in 4% SDS in 1×TBS buffer, and proteins were fragmented with trypsin. The biotin-labeled sections were separated using streptavidin beads (Thermo Fisher Scientific, Invitrogen), and only a peptide (Y+333 Da) in which biotin phenol of tyrosine residues was modified was selectively subjected to mass spectrometry.

As a result, the number of mitochondrial matrix proteins in the muscle and heart was confirmed to be 200 in the heart, 200 in the tibialis anterior muscle, and 251 in the soleus muscle, and as shown in FIG. 3, proteomes specifically expressed in muscle and the heart could be summarized according to their characteristics.

### Example 3. Mitochondrial TEM Images Using MAX to Mice

The heart, tibialis anterior, and soleus were each isolated from the transgenic mouse produced in Example 1, and fixed with 2.5% glutaraldehyde and 2% paraformaldehyde in a 0.1 M cacodylate solution (pH 7.0) at 4° C. for 1 hour. After washing, a 20 mM glycine solution was used to quench the unreacted aldehyde. DAB staining (Sigma-Aldrich) was performed for about 40 minutes until a light-brown stain was visible under a stereo microscope. DAB-stained tissues were post-fixed with 2% osmium tetroxide at 4° C. for 60 minutes, fixed with 1% uranyl acetate (Electron Microscopy Sciences) overnight, and then dehydrated with acetone (Sigma-Aldrich). Next, the sample was embedded using an Embed-812 embedding kit (Fisher Scientific) and subjected to a polymerization process in an oven at 60° C. The polymerized sample was sectioned (60 nm) using an ultramicrotome (UC7; Leica Microsystems, Germany), and sectioned samples were mounted on copper slot grids with a specimen support film. Sections were stained with UranyLess (Electron Microscopy Sciences) and observed using a Tecnai G2 transmission electron microscope (ThermoFisher, USA).

As a result, tissue-specific mitochondrial TEM images were obtained as shown in FIG. 4.

### Example 4. Analysis of Mitochondrial Matrix Proteome in Muscle Cells Using Floxed MAX-Tg Mice and Myf5-Cre Mice

In order to proceed with a cell-type specific proteomic analysis beyond the tissue-specific proteomic analysis in Example 1, the MTS-V5-APEX2 gene as produced in mice in the Examples was introduced into a Cre recombinase system. By adding a loxP-Stop codon-loxP gene in front of the MTS-V5-APEX2 gene, the MTS-V5-APEX2 gene was designed to be expressed only when the Cre recombinase is present (floxed MAX-Tg mice). Therefore, thereafter, by crossing floxed MAX-Tg mice with Cre mice suitable for a desired cell type, MTS-V5-APEX2 was allowed to be expressed only in a specific cell type.

Meanwhile, the expression of a muscle cell-specific MTS-V5-APEX2 gene was induced by crossing Myf5-Cre mice (JAX 007893, https://www.jax.org/strain/007893), which generate muscle-specific Cre recombinase, with floxed MAX-Tg mice.

Mass spectrometry samples were prepared and analyzed in the same manner as the experimental method of Example 2 to identify muscle cell-specific proteins.

Specifically, through the analysis, among the 200 proteins in the tibialis anterior muscle, a list of the top 20 most abundant proteins could be confirmed, and it could be confirmed that the majority of the proteins are proteins in the process of an OXPHOS complex (Atp5h, Atp5o, Atp5b, Atp5c1, Atp5a1, Ndufab1, Ndufa2, Cox7c, Uqcrc1, and Uqcrq) or TCA cycle (Idh3g, Mdh2, and Cs) (FIG. 5).

Although a specific part of the present disclosure has been described in detail, it will be obvious to a person with ordinary skill in the art that such a specific description is just a preferred embodiment and the scope of the present disclosure is not limited thereby. Accordingly, the substantial scope of the present invention will be defined by the appended claims and equivalents thereof.

1. A fusion protein in which a mitochondrial matrix targeting peptide and a proximity labeling enzyme are fused.

2. A recombinant expression vector or recombinant RNA comprising a sequence encoding the fusion protein of claim 1.

3. The recombinant expression vector or recombinant RNA of claim 2, wherein the recombinant expression vector or recombinant RNA comprises:
a first nucleotide sequence encoding a mitochondrial matrix targeting sequence;
a second nucleotide sequence encoding a proximity labeling enzyme; and
a promoter to which the first and second nucleotide sequences are operably linked.

4. The recombinant expression vector or recombinant RNA of claim 3, wherein the proximity labeling enzyme is APEX, APEX2 or TurboID.

5. A transgenic cell line or fertilized egg for producing a transgenic animal into which the recombinant expression vector or recombinant RNA of claim 2 has been introduced.

6. A transgenic animal in which a proximity labeling enzyme is specifically expressed in a mitochondrial matrix.

7. The transgenic animal of claim 6, wherein the animal is a mouse or rat.

8. A method for identifying a tissue-specific mitochondrial matrix protein, the method comprising the following steps:
(a) expressing a proximity labeling enzyme in the mitochondrial matrix of the tissue in the transgenic animal of claim 6;
(b) labeling a mitochondrial matrix protein with a phenol probe through a chemical reaction caused by a proximity labeling enzyme expressed in the mitochondrial matrix in the tissue of the transgenic animal; and
(c) identifying the labeled protein as a tissue-specific mitochondrial matrix protein.

9. The method of claim 8, wherein the proximity labeling enzyme is APEX, APEX2 or TurboID.

10. The method of claim 8, wherein in step (b), the tissue is isolated, and then subsequently treated with any one reagent selected from the group consisting of biotin phenol, desthiobiotin phenol, alkyne-phenol and azide-phenol, and hydrogen peroxide to label the mitochondrial matrix protein with a phenol probe.

11. The method of claim 8, wherein in step (c), the labeled protein is isolated and identified using streptavidin beads.

12. The method of claim 8, wherein the protein labeled in step (c) is identified by mass spectrometry.

13. A method for identifying a target protein in a drug-specific mitochondrial matrix, the method comprising the following steps:
(a) administering a drug to the transgenic animal of claim 6;
(b) expressing a proximity labeling enzyme in the mitochondrial matrix of the tissue in the drug-administered animal;
(c) labeling a mitochondrial matrix protein with a phenol probe through a chemical reaction caused by a proximity labeling enzyme expressed in the mitochondrial matrix in the tissue of the transgenic animal; and
(d) when the labeled protein has a significant change in expression level compared to a control, identifying the protein as a drug-specific mitochondrial matrix target protein.

14. The method of claim 13, wherein the proximity labeling enzyme is APEX, APEX2 or TurboID.

15. The method of claim 13, wherein in step (c), the tissue is isolated, and then subsequently treated with any one reagent selected from the group consisting of biotin phenol, desthiobiotin phenol, alkyne-phenol and azide-phenol, and hydrogen peroxide to label the mitochondrial matrix protein with a phenol probe.

16. The method of claim 13, wherein in step (d), the labeled protein is isolated and identified using streptavidin beads.

17. The method of claim 13, wherein the protein labeled in step (d) is identified by mass spectrometry.


SEQUENCE LISTING (6 sequences)

SEQ ID NO: 1 - protein, 24 aa, Artificial
  MTS amino acid sequence
MLATRVFSLVGKRAISTSVCVRAH

SEQ ID NO: 2 - protein, 14 aa, Artificial
  V5 amino acid sequence
GKPIPNPLLGLDST

SEQ ID NO: 3 - protein, 249 aa, Artificial
  APEX2 amino acid sequence
GKSYPTVSADYQDAVEKAKKKLRGFIAEKRCAPLMLRLAFHSAGTFDKGTKTGGPFGTIKHPAELAHSANNGLDIAVRLLEPLKAEFPILSYADFYQLAGVVAVEVTGGPKVPFHPGREDKPEPPPEGRLPDPTKGSDHLRDVFGKAMGLTDQDIVALSGGHTIGAAHKERSGFEGPWTSNPLIFDNSYFTELLSGEKEGLLQLPSDKALLSDPVFRPLVDKYAADEDAFFADYAEAHQKLSELGFADA

SEQ ID NO: 4 - protein, 290 aa, Artificial
  MTS-V5-APEX2 amino acid sequence
MLATRVFSLVGKRAISTSVCVRAHKDPGKPIPNPLLGLDSTGKSYPTVSADYQDAVEKAKKKLRGFIAEKRCAPLMLRLAFHSAGTFDKGTKTGGPFGTIKHPAELAHSANNGLDIAVRLLEPLKAEFPILSYADFYQLAGVVAVEVTGGPKVPFHPGREDKPEPPPEGRLPDPTKGSDHLRDVFGKAMGLTDQDIVALSGGHTIGAAHKERSGFEGPWTSNPLIFDNSYFTELLSGEKEGLLQLPSDKALLSDPVFRPLVDKYAADEDAFFADYAEAHQKLSELGFADA

SEQ ID NO: 5 - DNA, 21 bp, Artificial
  Primer F
gtcgacgagctcgtttagtga

SEQ ID NO: 6 - DNA, 21 bp, Artificial
  Primer R
aagaccgttgttagcgctgtg